(19) United States
(12) Patent Application Publication
(10) Pub. No.: US 2025/0032014 A1
(43) Pub. Date: Jan. 30, 2025

(54) CEREBRAL BLOOD FLOW MEASURING DEVICE USING NEAR-INFRARED RAYS

(71) Applicant: KOREA UNIVERSITY RESEARCH AND BUSINESS FOUNDATION, Seoul (KR)

(72) Inventors: Seung Ho PAIK, Seoul (KR); Seung Hyun LEE, Seoul (KR)

(21) Appl. No.: 18/708,982

(22) PCT Filed: Nov. 11, 2021
(86) PCT No.: PCT/KR2021/016469

§ 371 Date: May 9, 2024

(51) Int. Cl. A61B 5/1455; A61B 5/00

(52) U.S. Cl. CPC A61B 5/14553; A61B 5/6803; A61B 5/7264; A61B 5/742; A61B 2560/0462; A61B 2562/0247; A61B 2562/046; A61B 2562/164

(57) ABSTRACT

The present invention relates to a cerebral blood flow measuring device comprising: a flexible base configured to be transformed correspondingly to the shape of a head in close contact therewith; a plurality of light irradiation units provided on an installation surface, facing the head, of the flexible base; a plurality of light detection units provided on the installation surface; and an expansion unit configured to transform the flexible base. The cerebral blood flow measuring device using near-infrared spectroscopy according to the present invention can improve measurement accuracy by actively adjusting the pressure of the flexible base to thus bring the light irradiation units and the light detection units into close contact with each other according to the shape and size of the head.

In addition, research related to this patent is conducted with support from the National Research Foundation of Korea with funded by the government (Ministry of Science and ICT) in 2021 (NRF-2019R1C1C1011408).

### TECHNICAL FIELD

Research related to this patent was conducted with support from the Korea Health Industry Development Institute's Health and Medical Technology Research and Development Project (project number: HI14C3477) funded by the Ministry of Health and Welfare.

The present invention relates to a cerebral blood flow measuring device using near-infrared rays.

### BACKGROUND ART

Near-infrared spectroscopy is used to non-invasively secure information within a tissue by irradiating light in near-infrared areas region to the tissue to detect scattered and reflected light. An example of where the near-infrared spectroscopy is used, there is a method of measuring oxygen saturation in tissue/blood. The oxygen saturation is a value that represents tissue metabolism. It has been observed that when tissue metabolism becomes active, the oxygen saturation decreases, and conversely, when the tissue metabolism becomes slow, the oxygen saturation increases.

Recently, a method of using near-infrared spectroscopy has been used as a method of measuring a cerebral blood flow, but there was inconvenience in putting on and taking off the cerebral blood flow measuring device, and there was a problem in that accuracy was lowered depending on a shape and size of a head due to individual differences.

### DISCLOSURE

### Technical Problem

The present invention is directed to providing a cerebral blood flow measuring device using near-infrared spectroscopy.

### Technical Solution

One aspect of the present invention provides a cerebral blood flow measuring device, including: a flexible base configured to be deformed correspondingly to a shape of a head in close contact therewith; a plurality of light irradiation units provided on an installation surface, facing the head, of the flexible base; a plurality of light detection units provided on the installation surface; and an expansion unit configured to deform the flexible base.

The installation surface may have the plurality of light irradiation units and the plurality of light detection units arranged.

As the expansion unit expands, end portions of the plurality of light irradiation units and the plurality of light detection units may be configured to come into close contact with the head.

The expansion unit may be provided inside the flexible base and configured to expand the flexible base by receiving pressure from an outside.

The installation surface may be formed by extending long in left and right directions when the expansion unit is not expanded.

The installation surface may be configured to be away from the housing when the expansion unit is expanded.

The plurality of light detection units may be provided in greater numbers than the plurality of light irradiation units.

The plurality of light irradiation units may be provided in greater numbers than the plurality of light detection units.

The cerebral blood flow measuring device may further include a plurality of protrusions provided on the installation surface and formed to protrude at a predetermined height, in which the plurality of light irradiation units and the plurality of light detection units may each be provided at an end portion of the protrusion.

The cerebral blood flow measuring device may further include a housing configured to protect an outermost side of the flexible base.

The cerebral blood flow measuring device may further include a wearing part connected to one side of the housing or the flexible base and configured to be worn on the head.

The cerebral blood flow measuring device may further include a control unit configured to control the plurality of light irradiation units and the plurality of light detection units, and calculate oxygen saturation based on light detected by the light detection unit.

The cerebral blood flow measuring device may further include a display unit configured to be recognized by a person by sight or hearing.

When the oxygen saturation is greater than or equal to a predetermined threshold value, the control unit may control the display unit to display the oxygen saturation.

The cerebral blood flow measuring device may further include a pressure sensor configured to measure the pressure between the light irradiation unit and the light detection unit and the head surface, in which the control unit may calculate the oxygen saturation based on a value measured from the pressure sensor.

The light irradiation unit may be configured to irradiate light with a wavelength of 700 nm to 900 nm.

The control unit may function to calculate the oxygen saturation of the cerebral blood flow using near-infrared spectroscopy.

### Advantageous Effects

According to the cerebral blood flow measuring device using near-infrared spectroscopy according to the present invention, by actively adjusting the pressure of the flexible base to bring the light irradiation unit and light detection unit into close contact with each other according to the shape and size of the head, it is possible to improve the measurement accuracy.

In addition, the cerebral blood flow measuring device can be formed in the modular type and easily attached and detached, there by maximizing convenience.

### DESCRIPTION OF DRAWINGS

FIG. 1 is a perspective view of a cerebral blood flow measuring device according to an embodiment of the present invention.

FIG. 2 is a perspective view of the cerebral blood flow measuring device according to an embodiment of the present invention.

FIG. 3 is a cross-sectional view taken along line I-I′ of FIG. 1.

FIG. 4 is a partial perspective view illustrating an oxygen saturation measurement module array.

FIG. 5 is an enlarged exploded perspective view of a light irradiation unit and a light detection unit.

FIGS. 6A and 6B are conceptual diagrams illustrating an operating concept of the light irradiation unit and the light detection unit.

FIGS. 7A and 7B are diagrams illustrating a state of the cerebral blood flow measuring device according to an operation of an expansion unit.

FIG. 8 is a diagram illustrating an operating state of a notification unit.

FIG. 9 is a diagram illustrating a use state of the cerebral blood flow measuring device according to the present invention.

### MODES OF THE INVENTION

Hereinafter, a cerebral blood flow measuring device according to an embodiment of the present invention will be described in detail with reference to the accompanying drawings. In the description of the following embodiments, names of each component may be referred to by different names in the art. However, if there is functional similarity and identity therebetween, they can be viewed as equivalent configurations even if modified embodiments are adopted. In addition, symbols added to each component are described for convenience of description. However, the content illustrated in the drawings in which these symbols are written does not limit each component to the scope within the drawings. Likewise, even if an embodiment in which the configurations in the drawings are partially modified is adopted, it may be viewed as an equivalent configuration when there is functional similarity and identity. In addition, when it is recognized as a component that should naturally be included in light of the general level of technicians in the relevant technical field, the description thereof will be omitted.

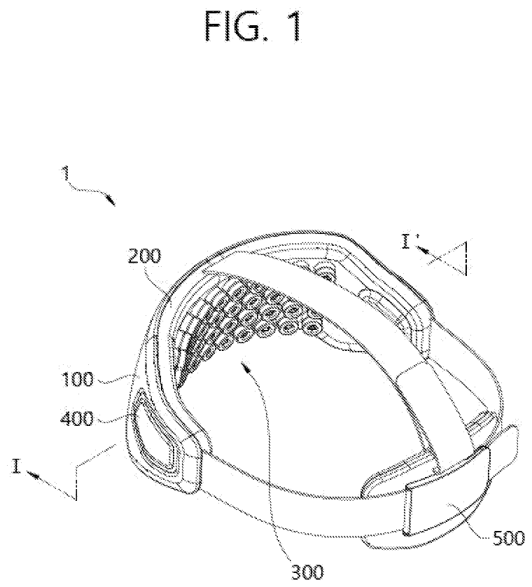

FIG. 1 is a perspective view of a cerebral blood flow measuring device 1 according to an embodiment of the present invention. The cerebral blood flow measurement device according to the present invention includes an oxygen saturation measurement array configured to measure blood flow, and is a device (head mounting device) configured to put on a head.

The cerebral blood flow measuring device 1 according to the present invention generally has a shape similar to that of a helmet, and includes a module capable of measuring oxygen saturation that may be provided in a portion that is in close contact with the head. Some elements of the cerebral blood flow measuring device 1 according to the present invention may be deformed to be in close contact with each other according to a shape and size of a user's head, allowing for personalized measurement. In addition, the user may take the cerebral blood flow measuring device 1 off after use, maximizing convenience of use.

In this embodiment, the oxygen saturation measurement module array, which will be described later, is configured to measure blood flow in a frontal lobe, but this is only an example, and the size and shape of the oxygen saturation measurement module array may be deformed so as to measure cerebral blood flow in at least one of the temporal lobe, parietal lobe, and occipital lobe.

Hereinafter, the configuration and function of the cerebral blood flow measuring device according to the present invention will be described in detail.

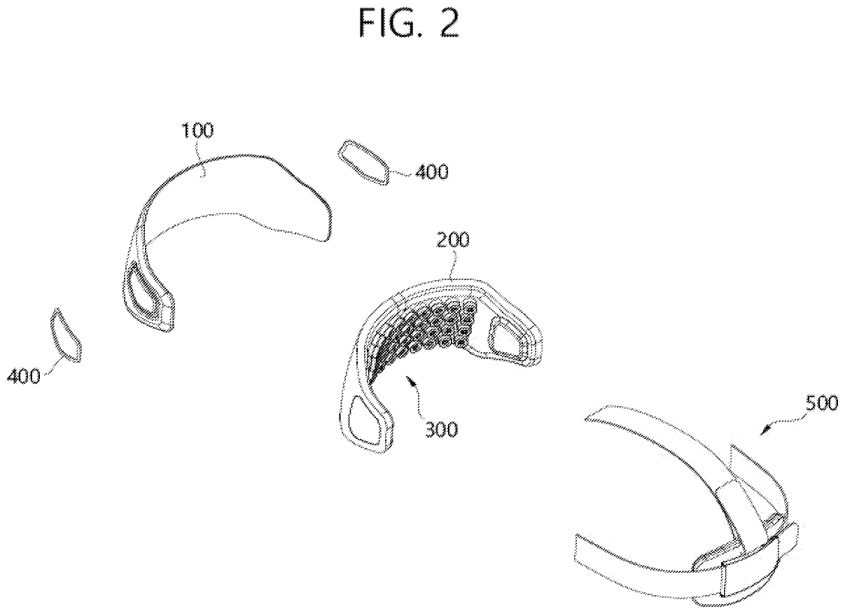

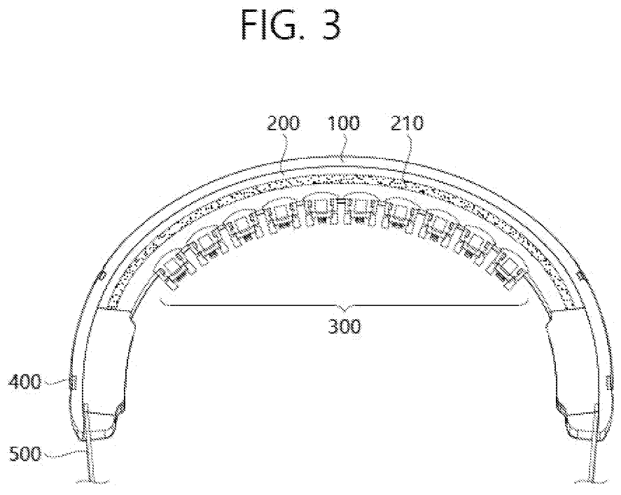

FIG. 2 is an exploded perspective view of the cerebral blood flow measuring device 1 according to an embodiment of the present invention, and FIG. 3 is a cross-sectional view taken along line I-I′ in FIG. 1.

Referring to FIGS. 2 and 3, the cerebral blood flow measuring device 1 according to an embodiment of the present invention may be configured to include a housing 100, a flexible base 200, an oxygen saturation measurement module array, an expansion unit 210, and a pressure sensor, a wearing part 500, a display unit 400, and a control unit (not illustrated).

The housing 100 is disposed on the outermost side and is configured to protect the flexible base 200, which will be described later. The housing 100 is, for example, configured in a curved shape and may be configured to be connected and fixed to a fastener, which will be described later. On one side of the housing 100, an installation space for the display unit 400 may be provided so that the display unit 400, which will be described later, may be provided.

The flexible base 200 is configured to be deformable according to external force, and one side facing the head may be provided with an installation surface 201. The flexible base 200 may be formed to extend to correspond to the area where cerebral blood flow is to be measured. When the flexible base 200 puts on the cerebral blood flow measuring device 1, one surface of the flexible base 200, which is in close contact with the user's head, may function as an installation surface 201. The installation surface 201 is configured to be provided with the oxygen saturation measurement module array, which will be described later.

The flexible base 200 is configured to be deformed according to an external force, that is, a force in which the oxygen saturation measurement module array is supported in close contact with the head. In addition, an expansion unit 210 may be provided inside the flexible base 200 so that it may be expanded as fluid flows in from the outside. The expansion unit 210 may be defined as a space that may receive fluid from the outside. The expansion unit 210 may be formed inside the flexible base 200 in response to an extension direction of the installation surface 201. Therefore, when the expansion unit 210 expands, the space between the housing 100 and the installation surface 201 is separated, and the installation surface 201 expands in the swelling direction. Ultimately, as the expansion unit 210 expands, the flexible base 200 is deformed, allowing the oxygen saturation measurement module array to come into close contact with the head. Meanwhile, a fluid port may be provided on one side of the flexible base 200 to allow fluid to be supplied or discharged from the outside. The fluid port may be in fluid communication with an external pump.

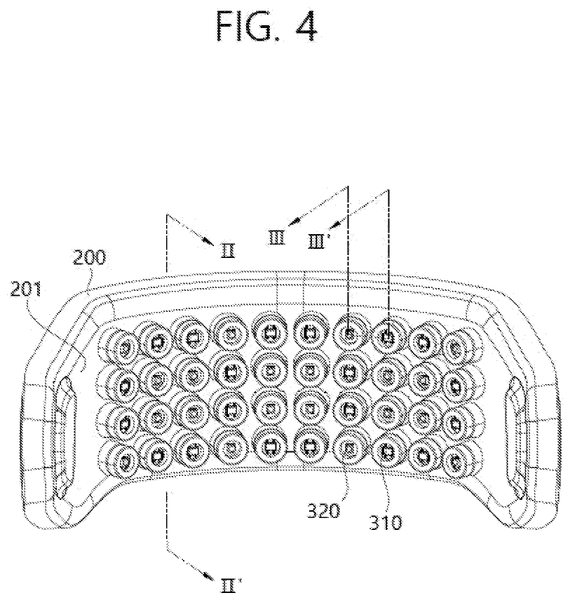

The oxygen saturation measurement module array is configured to measure the oxygen saturation of the cerebral blood flow. The oxygen saturation measurement module array may be configured to include a plurality of light irradiation units 310 and a plurality of light detection units 320. The light irradiation unit 310 and the light detection unit 320 may be arranged on the installation surface 201 in a predetermined pattern. In this case, the light irradiated from the light irradiation unit 310 may be detected by the plurality of light detection units 320. In addition, one light detection unit 320 may be configured to detect light irradiated from the plurality of light irradiation units 310. Meanwhile, the configuration of this oxygen saturation measurement module array will be described in detail later with reference to FIGS. 4 to 6B.

The pressure sensor (not illustrated) is configured to measure the pressure acting between the oxygen saturation detection module array 300 and the head. The pressure sensor may be formed in plurality and is provided on at least one of the installation surface 201, the light detection unit 320, or the light irradiation unit 310 to measure the pressure. The control unit may calculate the oxygen saturation using the value measured from the pressure sensor.

The wearing part 500 is configured to fix the cerebral blood flow measurement device to the head. The wearing part 500 may be configured of a fastening means such as a string connected to one side of the housing 100 or the flexible base 200. In this embodiment, the wearing part 500 is provided with strings connected to both left and right sides of the flexible base 200, and there is also an example in which it is configured of a T-shaped belt connected to the upper side. In addition, the wearing part 500 may be configured to include a component such as a buckle that may be decoupled for convenience of wearing.

The display unit 400 is configured to provide a notification based on the current oxygen saturation of cerebral blood flow while putting on the cerebral blood flow measuring device. The display unit 400 may be configured to be visually and/or audibly recognizable to people other than the user. Therefore, guardians or medical staff may immediately confirm that there is an abnormality in the cerebral blood flow.

The control unit (not illustrated) may be configured to perform the overall control of the cerebral blood flow measuring device 1. The control unit may be provided on one side of the flexible base 200 or the housing 100. The control unit controls the light irradiation unit 310 and the light detection unit 320, and may be configured to calculate the oxygen saturation of the cerebral blood flow based on the signal received from the light detection unit 320. The control unit may perform calculations to correct the oxygen saturation based on the value measured from the pressure sensor.

Hereinafter, the configuration of the oxygen saturation measurement module array will be described in detail with reference to FIGS. 4 to 6B.

FIG. 4 is a partial perspective view illustrating an oxygen saturation measurement module array.

Referring to FIG. 4, the oxygen saturation measurement module array may be configured to include the plurality of light irradiation units 310 and the plurality of light detection units 320. The plurality of light irradiation units 310 and the plurality of light detection units 320 may intersect or be arranged continuously in a certain pattern. One light detection unit 320 is configured to detect light irradiated from the plurality of light irradiation units 310, and the plurality of light detection units 320 are configured to detect the light irradiated from one light irradiation unit 310. Meanwhile, in this embodiment, the oxygen saturation measurement module is described as having a 4×10 array, but this is only an example, and may be modified into various arrangements.

The oxygen saturation measurement module array 300 is configured to irradiate light from the light irradiation unit 310, passes through the oxygen saturation measurement area, and receives the light scattered and/or reflected by the light detector 320 to calculate the blood flow oxygen saturation. When the oxygen saturation of the blood flow is lowered, the metabolic rate of brain cells may be assumed to have increased and the brain activity may be determined to be increasing. Conversely, when the oxygen saturation decreases, the metabolic rate of the brain cells may be assumed to decrease and the brain activity may be determined to decrease. It can be judged that the near-infrared spectroscopy is a method that non-invasively measures the change in concentration and optical coefficient of absorbing substances such as oxidized hemoglobin, reduced hemoglobin, and myoglobin present in human tissue, and uses near-infrared rays in the 700-900 nm band. Since the near-infrared rays have relatively small scattering and absorption within human tissue compared to other visible light bands, the light may reach deep, and using this, information may be obtained up to several centimeters deep within the human body. In this case, at least one of the plurality of light detection units 320 may detect the light irradiated from the light irradiation unit 310 to obtain the information related to the oxygen saturation

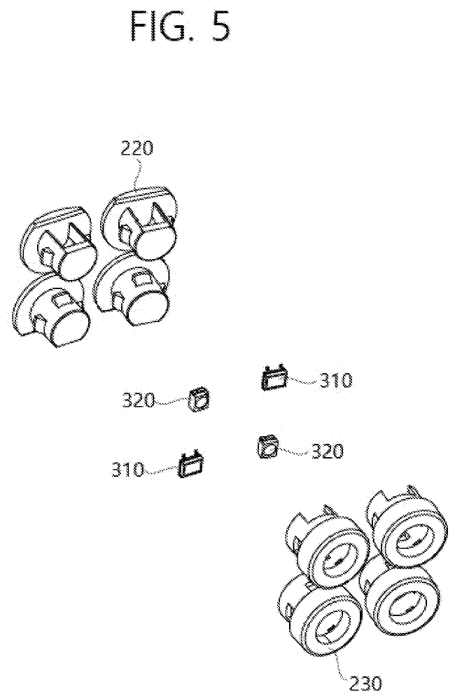

FIG. 5 is an enlarged exploded perspective view of the light irradiation unit 310 and the light detection unit 320.

Referring to FIG. 5, the light irradiation unit 310 and the light detection unit 320 may be provided on a protrusion 220 provided on the installation surface 201 of the flexible base 200. An end portion of the protrusion 220 may be provided with a flat surface so that the light irradiation unit 310 or the light detection unit 320 may be provided. The light irradiation unit 310 and the light detection unit 320 may each be protected by a cap 230. The cap 230 may be configured to be fixed to the protrusion 220, and a window may be installed in the central portion to allow light to pass through.

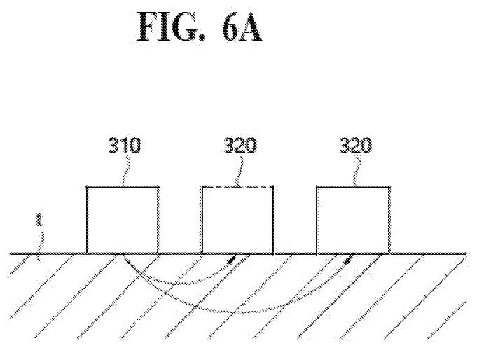

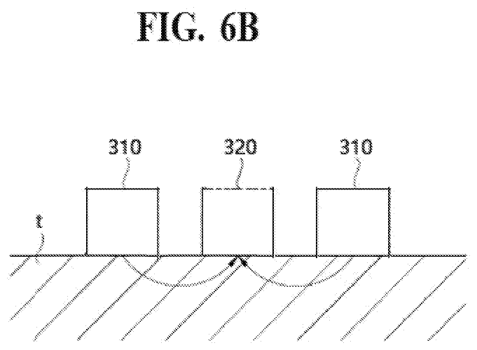

FIGS. 6A and 6B are conceptual diagrams illustrating the operating concept of the light irradiation unit 310 and the light detection unit 320.

Referring to FIG. 6A, the concept when cut along II-II′ in FIG. 4 is illustrated, and FIG. 6A illustrates the concept in which light irradiated from one light irradiation unit 310 is scattered and reflected from a tissue t and detected by each of the two light detection units 320.

Referring to FIG. 6B, the concept when cut along III-III′ in FIG. 4 is illustrated, and FIG. 6B illustrates the concept in which the light irradiated from the plurality of light irradiation units 310 is scattered and reflected from the tissue t and detected by one light detection units 320.

Meanwhile, the signal detected by the above-described light detection unit 320 may be transmitted to the control unit to perform calculation on the oxygen saturation.

In this case, the control unit may use an algorithm, for example, modified beer-Lambert law (MBLL), to derive meaningful analysis results based on the detected optical signal to calculate the oxygen saturation of the blood flow using the change in OxyHemoglobin concentration and the change in DeoxyHemoglobin concentration.

Meanwhile, the modified beer-Lambert law (MBLL) is as follows.

$$Δ {OD}^{λ} = - ln \frac{{I}_{Final}}{{I}_{Initial}} = ( {ε}_{{HbO}_{2}}^{λ} Δ [ {HbO}_{2} ] + {ε}_{Hbr}^{λ} Δ [ Hbr ] ) {B}^{λ} L$$

Here, ΔOD^{λ} denotes the optical density, and ε_{HbO}_{₂}^{λ}, εHbr^{λ} denote extinction coefficients, respectively, L denotes a source-detector separation, and B^{λ} denotes a differenctial pathlength factor

Here, an OxyHemoglobin concentration is as follows.

$$Δ [ {HbO}_{2} ] = \frac{{ε}_{Hbr}^{{λ}_{1}} \frac{Δ {OD}^{{λ}_{2}}}{{B}^{{λ}_{2}}} - {ε}_{Hbr}^{{λ}_{2}} \frac{Δ {OD}^{{λ}_{1}}}{{B}^{{λ}_{1}}}}{( {ε}_{Hbr}^{{λ}_{1}} {ε}_{{HbO}_{2}}^{{λ}_{2}} - {ε}_{Hbr}^{{λ}_{2}} {ε}_{{HbO}_{2}}^{{λ}_{1}} ) L}$$

In addition, a DeoxyHemoglobin concentration is as follows.

$$Δ [ Hbr ] = \frac{{ε}_{{HbO}_{2}}^{{λ}_{2}} \frac{Δ {OD}^{{λ}_{1}}}{{B}^{{λ}_{1}}} - {ε}_{{HbO}_{2}}^{{λ}_{1}} \frac{Δ {OD}^{{λ}_{2}}}{{B}^{{λ}_{2}}}}{( {ε}_{Hbr}^{{λ}_{1}} {ε}_{{HbO}_{2}}^{{λ}_{2}} - {ε}_{Hbr}^{{λ}_{2}} {ε}_{{HbO}_{2}}^{{λ}_{1}} ) L}$$

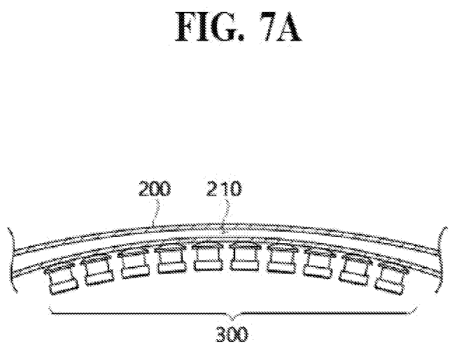

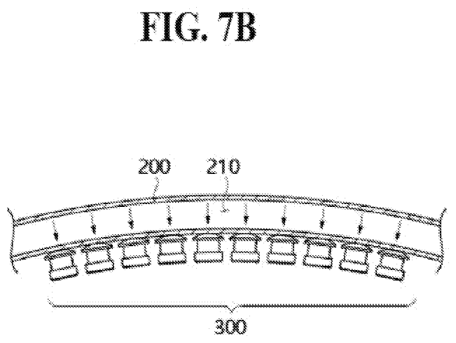

FIGS. 7A and 7B are diagrams illustrating a state of the cerebral blood flow measuring device according to an operation of the expansion unit 210.

Referring to FIG. 7A, the expansion unit 210 is not applied with pressure and has a certain elasticity in its initial state, and a position of the oxygen saturation detection module array 300 may be changed according to external force.

Referring to FIG. 7B, the expansion unit 210 is shown in a state where fluid is introduced from the outside and the expansion unit 210 swells. As the expansion unit 210 expands, the thickness of the flexible base 200 increases, that is, the gap between the housing 100 and the installation surface 201 increases, so the oxygen saturation detection module array 300 moves the head. Accordingly, the light irradiation unit 310 and the light detection unit 320 can be brought into close contact with the head with appropriate pressure, and may be deformed and adhered accordingly according to the curvature or asymmetrical shape of the head.

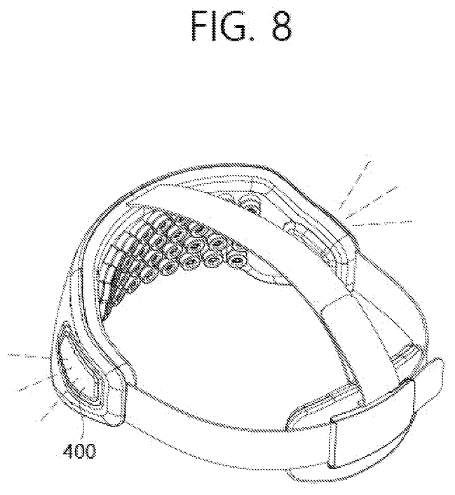

FIG. 8 is a diagram illustrating an operating state of a notification unit.

Referring to FIG. 8, the control unit may be configured to control the display unit 400 when it is determined that the metabolism of brain cells is not smooth in some areas as a result of calculating the oxygen saturation. That is, when it is determined that there is a problem with the user's cerebral blood flow, for example, when the oxygen saturation is below or above the threshold, the display unit 400 may operate to notify the user or guardian/medical staff. For example, when it is determined that there is an abnormality in the oxygen saturation of the cerebral blood flow, it is displayed in red or activates the speaker to make a sound so that guardians or medical staff may recognize abnormalities.

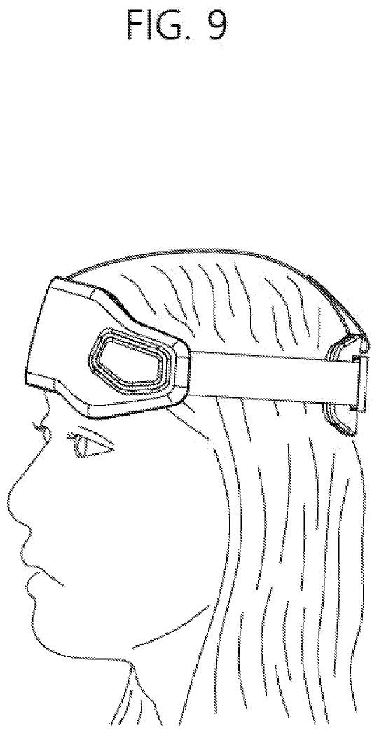

FIG. 9 is a diagram illustrating the use state of the cerebral blood flow measuring device 1 according to the present invention.

Referring to FIG. 9, the state of wearing the cerebral blood flow measuring device 1 according to the present invention is illustrated, and the user may take tests while putting on the cerebral blood flow measurement device or perform monitoring while going about their daily lives.

Meanwhile, although not illustrated, the cerebral blood flow measuring device 1 according to the present invention is configured to enable wireless communication with smart devices such as smart phones, laptops, PCs, and tablet PCs and measure and monitor the cerebral blood flow. In this case, when it is determined that there is a problem with cerebral blood flow, it may be configured to communicate with the smart device and notify the user, guardian, or medical staff.

As described above, the cerebral blood flow measuring device according to the present invention may actively control contact force by operating the expansion unit so that the plurality of light irradiation units and the light detection unit may be used in close contact with the head. Therefore, the inspection accuracy may be improved. Additionally, the cerebral blood flow measuring device is configured in a modular type and may be easily put on and taken off the head, maximizing convenience.

1. A cerebral blood flow measuring device, comprising:
a flexible base configured to be deformed correspondingly to a shape of a head in close contact therewith;
a plurality of light irradiation units provided on an installation surface, facing the head, of the flexible base;
a plurality of light detection units provided on the installation surface; and
an expansion unit configured to deform the flexible base.

2. The cerebral blood flow measuring device of claim 1, wherein the installation surface has the plurality of light irradiation units and the plurality of light detection units arranged.

3. The cerebral blood flow measuring device of claim 2, wherein as the expansion unit expands, end portions of the plurality of light irradiation units and the plurality of light detection units are configured to come into close contact with the head.

4. The cerebral blood flow measuring device of claim 3, wherein the expansion unit is provided inside the flexible base and is configured to expand the flexible base by receiving pressure from an outside.

5. The cerebral blood flow measuring device of claim 4, wherein the installation surface is formed by extending long in left and right directions when the expansion unit is not expanded.

6. The cerebral blood flow measuring device of claim 5, wherein the installation surface is configured to be away from the housing when the expansion unit is expanded.

7. The cerebral blood flow measuring device of claim 4, wherein the plurality of light detection units are provided in greater numbers than the plurality of light irradiation units.

8. The cerebral blood flow measuring device of claim 7, wherein the plurality of light irradiation units are provided in greater numbers than the plurality of light detection units.

9. The cerebral blood flow measuring device of claim 4, further comprising a plurality of protrusions provided on the installation surface and formed to protrude at a predetermined height, wherein the plurality of light irradiation units and the plurality of light detection units are each provided at an end portion of the protrusion.

10. The cerebral blood flow measuring device of claim 4, further comprising a housing configured to protect an outermost side of the flexible base.

11. The cerebral blood flow measuring device of claim 10, further comprising a wearing part connected to one side of the housing or the flexible base and configured to be worn on the head.

12. The cerebral blood flow measuring device of claim 4, further comprising a control unit configured to control the plurality of light irradiation units and the plurality of light detection units, and calculate oxygen saturation based on light detected by the light detection unit.

13. The cerebral blood flow measuring device of claim 12, further comprising a display unit configured to be recognized by a person by sight or hearing.

14. The cerebral blood flow measuring device of claim 13, wherein when the oxygen saturation is greater than or equal to a predetermined threshold value, the control unit controls the display unit to display the oxygen saturation.

15. The cerebral blood flow measuring device of claim 4, further comprising a pressure sensor configured to measure the pressure between the light irradiation unit and the light detection unit and the head surface, wherein the control unit calculates the oxygen saturation based on a value measured from the pressure sensor.

16. The cerebral blood flow measuring device of claim 4, wherein the light irradiation unit is configured to irradiate light with a wavelength of 700 nm to 900 nm.

17. The cerebral blood flow measuring device of claim 16, wherein the control unit functions to calculate the oxygen saturation of the cerebral blood flow using near-infrared spectroscopy.
